(19) United States
(12) Patent Application Publication
(10) Pub. No.: US 2019/0313943 A1
(43) Pub. Date: Oct. 17, 2019

(54) Biological Monitoring Device

(71) Applicant: AMI Inc., Kumamoto (JP)

(72) Inventor: Shinpei OGAWA, Kyoto (JP)

(21) Appl. No.: 16/474,069

(22) PCT Filed: Dec. 27, 2017
(86) PCT No.: PCT/JP2017/046881

§ 371 Date: Jun. 27, 2019

(30) Foreign Application Priority Data
Dec. 27, 2016 (JP) 2016-254258

(51) Int. Cl. A61B 5/08; A61B 5/113; A61B 8/14; A61B 7/00

(52) U.S. Cl. CPC A61B 5/08; A61B 5/113; A61B 7/04; A61B 7/003; A61B 8/14

(57) ABSTRACT

To identify exhalation and inhalation of a subject more accurately. A biological monitoring device (1) comprises: a monitoring unit (12) which monitors changes over time in the depth of feature points at one or a plurality of locations within a living body, on the basis of a received signal received from an ultrasonic sensor (2); and an exhalation/inhalation identifying unit (13) which identifies exhalation or inhalation on the basis of information relating to the depth of the feature points. Contraction and expansion of the lungs, in other words exhalation and inhalation, can be identified by applying ultrasound waves from between the ribs of a subject to monitor movements of biological tissue which moves together with the lungs.

### TECHNICAL FIELD

The present invention relates to a living body monitoring device for monitoring living body information such as respiration and beat of a subject.

### BACKGROUND ART

One of the sounds heard with a stethoscope is lung sounds that are emitted from the lungs during respiration. This lung sound is generally classified as illustrated in FIG. 7. Lung sounds are broadly classified into breath sounds and adventitious sounds, and breath sounds and adventitious sounds are further subclassified. Here, in order to determine the type of adventitious sounds, it is necessary to discriminate whether the adventitious sounds originates from “expiration” or “inspiration”.

### CITATION LIST

### Patent Literatures

Patent Literature 1: JP 2014-61223 A

### SUMMARY OF INVENTION

### Technical Problem

Now, in the case of face-to-face auscultation, it is easy to discriminate between expiration and inspiration since it is possible to visually determine a respiratory condition. However, in recent years, with advances in technology, digitization of auscultation equipment has progressed, and it is predicted that automatic diagnostic technology using IoT and machine learning, etc. will further spread in the future. In the case of automatic diagnosis, it is required to discriminate between expiration and inspiration instantaneously without facing a subject. However, since there is almost no difference between lung sounds at the time of expiration and at the time of inspiration, it is difficult to discriminate between expiration and inspiration only by analyzing information regarding lung sounds (sone, dB, Hz, etc.). Thus lung sounds cannot be discriminated automatically by auscultation sounds if it is impossible to distinguish between expiration and inspiration.

Further, for example, Patent Literature 1 discloses a vital signs instrument which can simultaneously obtain information regarding a disease site and the severity and shorten a time taken to diagnose a respiratory disease by simultaneously performing measurement of respiratory rate and local breath sounds. Although the instrument of Patent Literature 1 is considered to be capable of counting the respiratory rate by detecting vibration based on the breathing exercise of a subject, it has not been possible to identify whether breath sounds are caused by expiration or by inspiration. Thus, it is difficult to discriminate between expiration and inspiration using the vibration of breathing exercise, and in order to achieve that, it is necessary to consider other objective indicators.

In addition, respiratory rate is one of the significant vital signs as well as blood pressure, pulse and temperature. However, although it is known to detect the vibration generated by the movement of the chest and estimate respiratory rate as in the instrument of Patent Literature 1, such observation from a body surface is affected by other movement, etc. of the subject, and it is difficult to accurately measure only the number of breathing exercise.

Therefore, one of the objects of the present invention is to more accurately discriminate the subject's expiration and inspiration. Further, another object of the present invention is to more accurately diagnose lung sound of the subject based on the discrimination result between expiration and inspiration.

### Solution to Problem

The inventor of the present invention has found that biological tissues such as pleura, muscle, adipose tissue, and tendon located on the body surface side relative to the lung are displaced in conjunction with the lung in accordance with lung contraction and expansion. In addition, the present inventor has obtained knowledge that although it is difficult to confirm the detailed position of the lung by ultrasonic echo, it is possible to identify lung contraction and expansion, that is, expiration and inspiration by applying ultrasonic wave from between the ribs of the subject to monitor the movement of biological tissues in conjunction with the lung. Then, based on the above findings, the present invention has been completed with the idea that the problems of the prior art can be solved. Specifically, the present invention has the following configuration.

The present invention relates to a living body monitoring device. The living body monitoring device of the present invention includes a monitoring unit and a respiration identifying unit. The monitoring unit monitors a change over time in depth of one or more feature points in a living body based on a signal received from an ultrasonic sensor. The depth of the feature point is a distance from a living body surface (ultrasonic sensor) to the feature point. Further, the in-vivo feature point is basically a biological tissue located between a living body surface in contact with an ultrasonic sensor and the lung. Examples of in-vivo feature points include pleura, muscle, adipose tissue, and tendon located on the living body surface side of the lung. Now that, the lung may be included in the feature points of monitoring. However, it is usually difficult to detect the location (depth) of the lungs using ultrasonic wave because the lungs are almost full of air. In addition, the in-vivo feature points may be a part other than the lung, pleura, muscle, adipose tissue, and tendon described above. That is, it is also possible to determine a part or space that cannot be detected by ultrasonic wave as a feature point and to monitor the position (depth) of the part or space. The respiration identifying unit identifies expiration or inspiration based on information regarding the depth of the feature point. That is, the fact that the depth of the feature point is shallow means that the distance between the feature point and the living body surface (ultrasonic sensor) is short by the expansion of the lungs. On the contrary, the fact that the depth of the feature point is deep means that the distance between the feature point and the living body surface (ultrasonic sensor) is long by the contracting of the lungs. Therefore, if the information on the depth of the feature point is used, the respiration identifying unit can identify the timing of the subject's expiration and/or inspiration.

As described above, in the present invention, the depth of the in-vivo feature point that can be detected by the ultrasonic sensor is used as an objective index for discriminating between expiration and inspiration. In this way, by determining contraction and expansion of the lungs, it is possible to detect expiration and inspiration easily and accurately. Further, the fact that expiration and inspiration can be accurately discriminated means that respiration rate (respiration rate per unit time) can also be accurately detected. For this reason, the living body monitoring device of the present invention can be used to count the respiratory rate in addition to the discrimination of expiration and inspiration.

It is preferable that the living body monitoring device of the present invention further includes a synthesis unit. A synthesis unit generates ultrasonic data indicating the reception intensity of reflected ultrasonic wave by using the signal received from the ultrasonic sensor. In this case, the feature points are determined based on the reception intensity in the ultrasonic data. Examples of the ultrasonic data include image data including so-called A mode (Amplitude mode), B mode (Brightness mode), and M mode (Motion mode). Among these, in particular, the ultrasonic data is preferably in the B mode or M mode in which the reception intensity of reflected ultrasonic wave is represented by luminance.

As in the above configuration, creating ultrasonic data using received signals from the ultrasonic sensor makes it easy to determine feature points in a living body, and also makes it easy to distinguish the feature points from other sites. Consequently, more accurate monitoring is possible. Further, the reflected waves reflected at the pleura, muscles, tendons, and adipocytes are common in that the reception intensity at the ultrasonic sensor is high, and when the reception intensity is represented by the B mode or M mode, the luminance values of those sites become high. That is, it is possible to accurately grasp respiration condition by using the motion of the living body site having high reception intensity and luminance of such reflected waves as an index for expiration/inspiration discrimination. It is an example to make movement of biological tissues having high luminance into an index, and it is also possible to determine expiration and inspiration using biological tissues or space having low luminance as an index. However, the feature points are not limited to sites other than the lung. In the case of normal lungs, multiple reflection images called A-line can be seen inside the lungs. Since A-line is brighter than others, it is possible to monitor A-line inside the lungs as the feature points.

In the present invention, preferably, the monitoring unit determines the shallowest depth and the deepest depth of the feature point in one respiratory cycle. The shallowest depth and the deepest depth obtained by the monitoring unit are stored in a storage unit. The monitoring unit can also calculate the depth of respiration by calculating the difference between the shallowest depth and the deepest depth.

As described above, by automatically calculating the shallowest depth or the deepest depth of the feature point or the depth of respiration, for example, it can be used for diagnosis of agonal respiration (mandibular respiration, nasal alar respiration, gasp respiration). That is, although the agonal respiration has apparently a large respiratory movement, it is a state where respiration in the lungs is shallow, and the lungs hardly contract. Conventionally, there has been no index indicating the depth of respiration by the lungs, and therefore a prompt diagnosis of agonal respiration has been difficult. However, if the present invention is applied, it is possible to objectively measure the depth of respiration and to contribute to the early detection of the agonal respiration.

In the present invention, it is preferable that the respiration identifying unit identifies the period until the feature point displaces from the shallowest depth to the deepest depth as expiration and the period until the feature point displaces from the deepest depth to the shallowest depth as inspiration. By doing this, it is possible to clearly distinguish between expiration and inspiration.

In the present invention, it is preferable that the monitoring unit obtains the expiratory time that is a period until the feature point displaces from the shallowest depth to the deepest depth and the inspiratory time that is a period until the feature point displaces from the deepest depth to the shallowest depth The shallowest depth and the deepest depth obtained by the monitoring unit are stored in a storage unit.

As described above, by measuring the expiratory time and the inspiratory time, it can be used, for example, for diagnosis of asphyxiation, tongue base subsidence, asthma and the like. Asphyxiation and tongue base subsidence are characterized by the induction of inspiratory prolongation and strider at the time of inspiration, compared with the healthy state. In addition, asthma is characterized in that it causes prolongation of expiration and wheeze at the time of expiration, compared with the healthy state. Therefore, if the subject's expiratory time and inspiratory time are monitored using the present invention, when the above symptoms occur, the symptoms can be detected at an early stage.

It is preferable that the living body monitoring device of the present invention further includes a lung sound analysis unit. The lung sound analysis unit identifies the lung sound at the time of expiration and the lung sound at the time of inspiration based on the lung sound data received from the lung sound sensor and the information on expiration and inspiration identified by the respiration identifying unit. That is, there is almost no difference between lung sounds at the time of expiration and at the time of inspiration, and therefore, by analyzing lung sound data from the lung sound sensor, it is difficult to discriminate whether the lung sounds were generated at the time of expiration or at the time of inspiration. Therefore, by linking the lung sound data obtained from the lung sound sensor with the information regarding expiration and inspiration identified by the respiration identifying unit, the lung sound at the time of expiration and the lung sound at the time of inspiration can be determined accurately, easily and automatically.

As described above, the living body monitoring device according to the present invention can accurately distinguish between the lung sound at the time of expiration and the lung sound at the time of inspiration, by using with an auscultation sound (lung sound sensor) which can obtain lung sounds as digital signals. Therefore, classification of lung sounds is facilitated based on the index as indicated in FIG. 7, and as a result, diagnosis of various diseases such as asthma and asphyxiation can be supported.

It is preferable that the living body monitoring device of the present invention further includes a lung sound determination unit. The lung sound determination unit classifies sound components included in lung sound data based on the lung sound at the time of expiration and/or the lung sound at the time of inspiration identified by the sound absorption analysis unit. The method of classifying lung sounds is not particularly limited. For example, classification may be performed on the basis of information such as whether lung sounds are generated at the time of expiration or at the time of inspiration, or information such as frequency of lung sounds. As an example, lung sounds are classified according to the classification table described in FIG. 7.

As in the above-described configuration, when the living body monitoring device includes the lung sound determination unit, sound components included in the lung sound data received from the lung sound sensor can be automatically classified. For this reason, a medical worker can more accurately diagnose various diseases such as asthma and asphyxia by using this living body monitoring device.

In the living body monitoring device according to the present invention, the respiration identifying unit preferably further identifies the timing of apnea based on information on the depth of the feature point. For example, if the depth of the feature point is substantially constant, and the time that the depth does not change continues for a fixed time, the respiration identifying unit may determine that the period is apnea. In this case, it is preferable that the living body monitoring device of the present invention further includes a heart sound analysis unit. The heart sound analysis unit extracts heart sounds during apnea identified by the respiration identifying unit from heart sound data received from the heart sound sensor.

To evaluate the heart sounds of subjects, heart murmur of heart valve disease is mainly evaluated. However, lung sounds are a problem when heart sounds are evaluated. That is, if the subject breathes deeply while auscultating a heart sound, the heart sound and a lung sound overlap, and it is difficult to evaluate whether the noise included in the auscultation sound is a heart murmur or a simple lung sound. Therefore, as in the above configuration, by extracting heart sound components from the heart sound data at the timing when the subject is apnea, it is possible to exclude lung sound components of the subject to accurately evaluate the heart sound and the heart murmur.

### Advantageous Effects of Invention

According to the present invention, the subject's expiration and inspiration can be discriminated more accurately. In addition, it is also possible to diagnose lung sounds of the subject more accurately based on the discrimination result of expiration and inspiration.

### BRIEF DESCRIPTION OF DRAWINGS

FIG. 1 is a schematic view illustrating an outline of a living body monitoring system.

FIG. 2 is a block diagram illustrating an example of the configuration of the living body monitoring system.

FIG. 3 illustrates an example of ultrasonic data.

FIG. 4 illustrates an example of a method for identifying expiration and inspiration based on ultrasonic data (M mode).

FIG. 5 illustrates an example of a method of linking ultrasonic data and lung sound data.

FIG. 6 illustrates an example of a method of linking ultrasonic data and heart sound data.

FIG. 7 illustrates an example of a classification table of lung sounds.

### DESCRIPTION OF EMBODIMENTS

An embodiment of the present invention will be described below using the drawings. The present invention is not limited to the embodiments described below and includes those appropriately modified by a person skilled in the art from the following embodiments within the obvious scope.

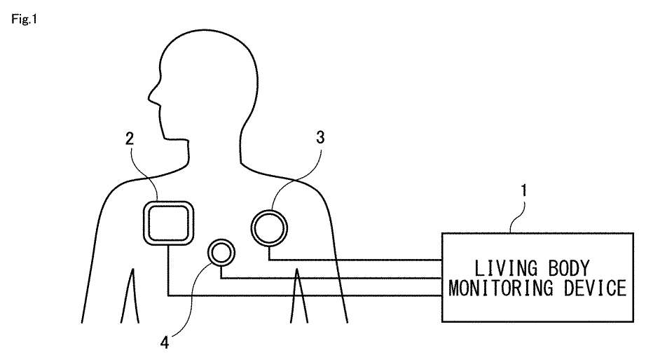

FIG. 1 shows an outline of a living body monitoring system. As illustrated in FIG. 1, the living body monitoring system according to the present embodiment includes a living body monitoring device 1, an ultrasonic sensor 2, a lung sound sensor 3, and a heart sound sensor 4. The living body monitoring system may include at least the living body monitoring device 1 and the ultrasonic sensor 2, and the other lung sound sensor 3 and heart sound sensor 4 are optional components. The ultrasonic sensor 2, the lung sound sensor 3, and the heart sound sensor 4 are each connected to the living body monitoring device 1, and according to the control by the living body monitoring device 1, they acquire various information (sensing information) and transmit the acquired information to the living body monitoring device 1. The various sensors 2, 3, and 4 may be connected to the living body monitoring device 1 by wire or wirelessly. In the case of wireless connection, the various sensors 2, 3, and 4 may be connected to the living body monitoring device 1 through the Internet, or may be connected to the living body monitoring device 1 by near field communication by a known standard such as Wi-Fi (registered trademark) or Bluetooth (registered trademark).

An example of the ultrasonic sensor 2 is an ultrasonic probe. The ultrasonic sensor 2 (ultrasonic probe) transmits ultrasonic waves into a living body, receives and measures its reflected wave. The ultrasonic sensor 2 may be configured by, for example, a sensor array as a transmitting/receiving unit in which a plurality of ultrasonic transducers are two-dimensionally arranged. At the time of ultrasonic measurement, the ultrasonic sensor 2 is positioned on the living body surface of a subject 7. Specifically, the ultrasonic sensor 2 is positioned directly above the pleura such that ultrasonic waves can be applied to the lungs through the intercostal of the subject. The ultrasonic sensor 2 transmits a pulse signal or burst signal of ultrasonic waves of MHz to several tens of MHz from the intercostal towards the lungs, and receives reflected waves including reflections from the lungs and reflections from the living body tissues such as the pleura, muscles, adipose tissue, and tendons located between the living body surface and the lungs. The living body monitoring device 1 generates reflected wave data related to the in-vivo structure of the subject by amplifying and processing the signal received from the ultrasonic sensor 2. The ultrasonic measurement is repeatedly performed at a predetermined measurement cycle (for example, a frame rate of 300 to 500 per second).

For the lung sound sensor 3 and the heart sound sensor 4, a known sound sensor can be used, which inputs sound generated in a living body as vibration and converts the input vibration into voltage to obtain a sound signal. The lung sound sensor 3 and the heart sound sensor 4 amplify sound signals and perform signal processing to convert analog signals into digital data, and input lung sound data and heart sound data to the living body monitoring device 1. The amplification processing and signal processing of the acoustic signal may be performed by the living body monitoring device 1 instead of the lung sound sensor 3 and the heart sound sensor 4 respectively. As will be described later, the living body monitoring device 1 links lung sound data received from the lung sound sensor 3 or heart sound data received from the heart sound sensor 4 to ultrasonic data obtained from the ultrasonic sensor 2 to analyze the subject's lung sounds or heart sounds.

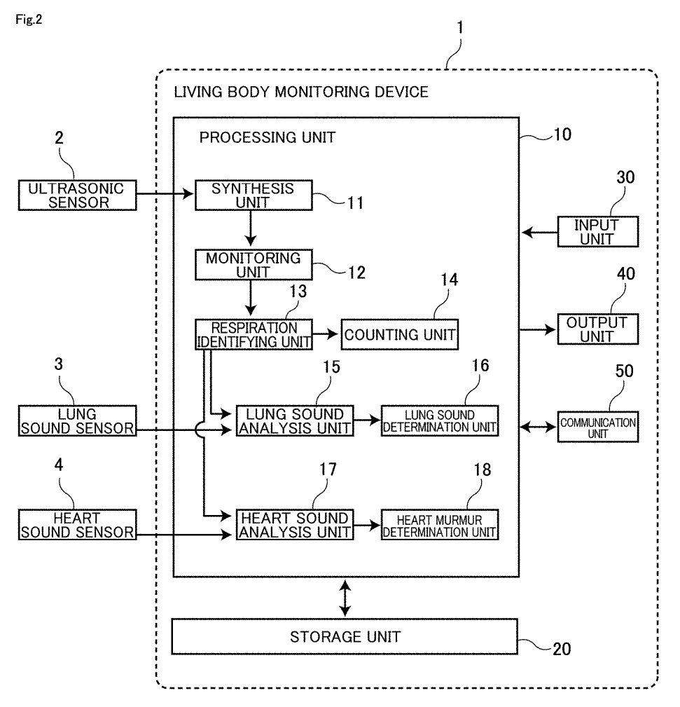

FIG. 2 is a block diagram illustrating the configuration of the living body monitoring system, in particular, the configuration of the living body monitoring device 1. As indicated in FIG. 2, the living body monitoring device 1 includes a processing unit 10, a storage unit 20, an input unit 30, an output unit 40, and a communication unit 50.

The processing unit 10 is a control arithmetic device that entirely controls each element of the living body monitoring device 1, and can be realized by a processor such as a CPU or a GPU. The processing unit 10 reads a program stored in the storage unit 20 and controls other elements in accordance with the program. Further, the processing unit 10 can appropriately write and read out calculation results according to the program in the storage unit 20.

The storage unit 20 is an element for storing information to be used for calculation processing and the like in the processing unit 10. The storage function of the storage unit 20 can be realized by, for example, nonvolatile memories such as HDD and SDD. In addition, the storage unit 20 may have a function as a memory for writing or reading progress of the calculation processing by the processing unit 10 or the like. The memory function of the storage unit 20 can be realized by volatile memory such as RAM or DRAM. Further, the storage unit 20 stores a program that causes a general-purpose computer or a portable information communication terminal to function as the living body monitoring device 1 according to the present invention. This program may be downloaded to the living body monitoring device 1 via the Internet or may be preinstalled on the living body monitoring device 1. When a program for this system is activated according to a command from the user, processing according to this program is executed by the processing unit 10.

The input unit 30 is an element for receiving an input of information from the user to the living body monitoring device 1. The information input through the input unit 30 is transmitted to the processing unit 10. The input unit 30 can employ various input devices used in known computers. Examples of the input unit 30 include, but are not limited to, a touch panel, a button, a cursor, a microphone, a keyboard, and a mouse. Further, a touch panel included in the input unit 30 may form a touch panel display together with a display included in the output unit 40.

The output unit 40 is an element for outputting various types of information to the user. The information subjected to calculation processing in the processing unit 10 is output by the output unit 40. The output unit 40 can employ various external output devices used in known computers. Examples of the output unit 40 include, but are not limited to, a display, a speaker, a light, and a vibrator. As described above, the display may be a touch panel display.

The communication unit 50 is an element for exchanging information between the living body monitoring device 1 and another device through a communication line such as the Internet, for example. For example, the communication unit 50 can also transmit the calculation result of the processing unit 10 and the information stored in the storage unit 20 to a server device that manages and controls various types of information through the Internet or can also receive various types of information from the server device. Since the living body monitoring device 1 includes the communication unit 50, the living body information of the subject can be remotely monitored.

Subsequently, the functional configuration of the processing unit 10 will be specifically described. As indicated in FIG. 2, in the present embodiment, the processing unit 10 includes a synthesis unit 11, a monitoring unit 12, a respiration identifying unit 13, a counting unit 14, a lung sound analysis unit 15, a lung sound determination unit 16, a heart sound analysis unit 17, and a heart murmur determination unit 18.

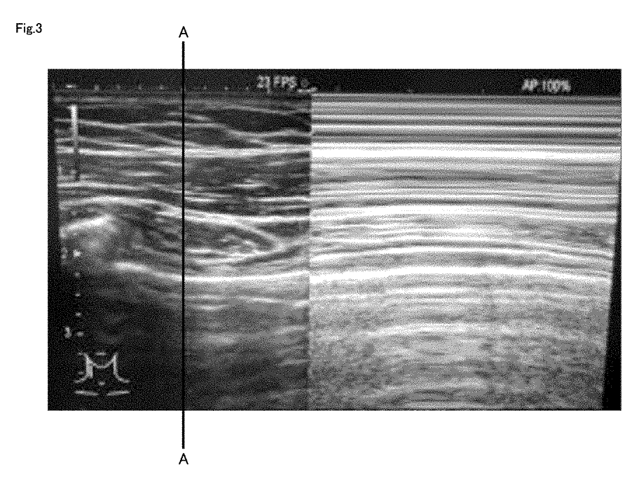

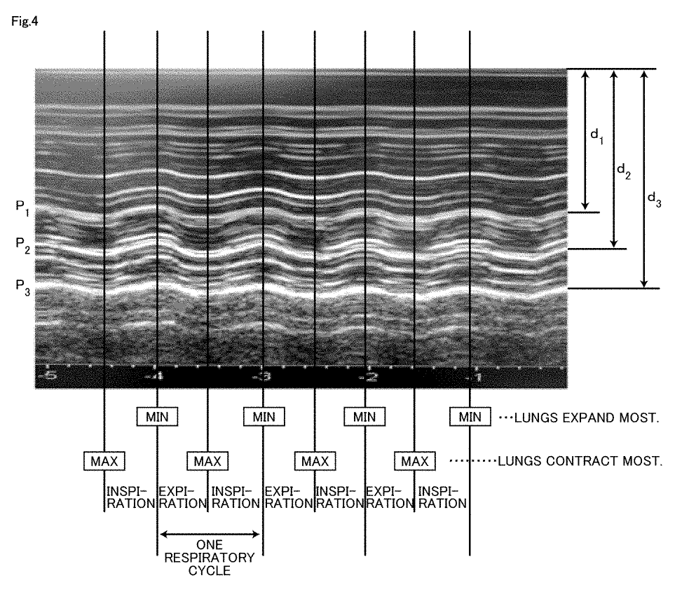

The synthesis unit 11 generates ultrasonic data indicating the reception intensity of reflected ultrasonic wave by using the signal received from the ultrasonic sensor 2. Now that, in the living body monitoring device 1, after amplification processing and signal processing are performed on a received signal from the ultrasonic sensor 2, the digital signal after the processing may be input to the synthesis unit 11. Examples of ultrasonic data include image data including so-called A mode, B mode, and M mode. In the A mode, the amplitude of a reflected wave (A mode image) is displayed, with the first axis as the distance in the depth direction (Z direction) from the predetermined living body surface position and the second axis as the received signal intensity of the reflected wave. The A mode displays the amplitude or envelope of an echo signal and can only express information in a specific direction, but it is easy to identify the position of a reflector. The B mode displays a two-dimensional image (B-mode image) of an in-vivo structure visualized by converting the amplitude (A-mode image) of reflected wave obtained while scanning the living body surface position into a luminance value. The M mode is a method of displaying temporal changes in the position of a reflector. In the M mode, beam scanning is not performed, transmission and reception are repeated in a fixed direction, an echo signal is represented by a single bright line modulated in luminance, and the display position of the bright line is moved horizontally in parallel according to the passage of time to display a temporal change, that is movement, of a reflection position. Among these, in particular, the ultrasonic data is preferably in the B mode or M mode in which the reception intensity of reflected ultrasonic wave that is represented by luminance. FIG. 3 illustrates an example of a display screen of ultrasonic data. In FIG. 3, the left screen indicates ultrasonic data of the B mode, and the right screen indicates ultrasonic data of the M mode. In the M mode, the temporal change of the site indicated by line A-A in the left screen is indicated. The synthesis unit 11 stores generated ultrasonic data in the storage unit 20.

The monitoring unit 12 determines one or more feature points in the living body based on the ultrasonic data generated by the synthesis unit 11, and monitors a temporal change in depth from the living body surface for the feature points. Examples of in-vivo feature points include pleura, muscle, adipose tissue, and tendon located on the living body surface side of the lung. Now that, the lung may be included in the feature points to be monitored. Since the A-line in the lung is brighter than others, it is possible to monitor this A-line as a feature point. In addition, it is preferable to exclude blood vessels and blood vessel walls from the feature points. Since blood vessels and the like are contracted and vibrated finely regardless of the subject's respiration due to blood flow and pulsation, these are unsuitable as monitoring targets for discrimination of expiration and inspiration. If the ultrasonic data is in the A mode, the monitoring unit 12 may determine one or more regions with high received intensity of the reflected wave as feature points and monitor the feature points. If the ultrasonic data is in the B mode or the M mode, the monitoring unit 12 may determine one or more sites with high luminance values on the image as feature points and monitor the feature points. The M-mode is most suitable for monitoring such temporal change of the depth of feature points. Therefore, the case where ultrasonic data is the M mode will be explained below as an example.

FIG. 3 illustrates an example of M-mode ultrasonic data. In the example of FIG. 3, among ultrasonic data, three points having the highest luminance value are set as feature points, and the depth of the feature points is monitored. At high luminance values, living tissues that move in conjunction with expansion and contraction of the lung, such as pleura, muscles, adipose tissue, and tendons, are positioned. Therefore, by monitoring the displacement of the living tissue with such a high luminance value, it is possible to estimate the extension and contraction of the lung indirectly. In FIG. 3, three points selected as feature points are indicated by reference numerals P₁, P₂, and P₃, respectively, and the depths of the feature points P₁ to P₃ are indicated by d₁, d₂, and d₃, respectively. Each of the feature points P₁ to P₃ is displaced up and down as the lungs contract. For this reason, the depth of each feature point periodically repeats to be shallowly displaced and deeply displaced with respiration.

The monitoring unit 12 sequentially acquires depth values for each feature point, and stores the acquired depth values in the storage unit 20. For example, the monitoring unit 12 may store only the shallowest depth value and the deepest depth value of each feature point in one respiratory cycle, or in addition to the shallowest depth and the deepest depth, a value of the intermediate depth therebetween may also be stored. Note that one respiratory cycle is a period from a certain shallowest depth to the next shallowest depth (or a period from a certain deepest depth to the next deepest depth). For example, when performing automatic diagnosis, remote diagnosis, or machine learning for automatic diagnosis, it is important to distinguish between expiratory time and inspiratory time, but in that case, it is particularly important to quantify the reference information to distinguish between expiration and inspiration. According to the present invention, the depth from the living body surface is acquired as a numerical value for one or a plurality of feature points, and expiration and inspiration are distinguished based on the depth value. In this way, by tracking the change in depth value, it is possible to automatically determine whether it is in the expiratory time or in the inspiratory time at this moment.

Further, the monitoring unit 12 calculates the difference between the shallowest depth and the deepest depth in one respiratory cycle to obtain the depth of respiration, and stores the information on the depth of the respiration in the storage unit 20. Further, the depth of respiration (difference value) obtained by the monitoring unit 12 can be displayed by a display. Thus, by automatically calculating the shallowest depth or the deepest depth of a feature point or the depth of respiration, for example, it can be used for diagnosis of agonal respiration (mandibular respiration, nasal alar respiration, agonal respiration).

Furthermore, the monitoring unit 12 obtains the expiratory time that is a period until the feature point displaces from the shallowest depth to the deepest depth and the inspiratory time that is a period until the feature point displaces from the deepest depth to the shallowest depth within one respiratory cycle. Information on the expiratory time and the inspiratory time may be stored in the storage unit 20 as required. In addition, the expiratory time and the inspiratory time determined by the monitoring unit 12 can be displayed by the display. Thus, by measuring the expiratory time and the inspiratory time, it can be used, for example, for diagnosis of asphyxiation, tongue base subsidence, asthma, chronic obstructive pulmonary disease (COPD), bronchitis and the like.

The respiration identifying unit 13 identifies expiration and inspiration based on the depth information of each feature point monitored by the monitoring unit 12. That is, the state in which the depth of each feature point is the shallowest means a state in which the lungs expand most. Conversely, the state in which the depth of each feature point is the deepest means a state in which the lungs contract most. For this reason, the respiration identifying unit 13 identifies the period until the feature point displaces from the shallowest depth to the deepest depth in one respiratory cycle as “at the time of expiation”, and identifies the period until the feature point displaces from the deepest depth to the shallowest depth as “at the time of inspiration”.

It is preferable that when the monitoring unit 12 monitors temporal changes in depth of a plurality of feature points, the respiration identifying unit 13 performs identification of expiration and inspiration using composite depth information of a plurality of feature points for calculation. For example, when the feature points are three points P₁, P₂, and P₃, a total value of the depths of the feature points P₁ to P₃ is obtained, and a period from the minimum value of the total value to the maximum value may be identified as “at the time of expiration”, and the period from the maximum value of the total value to the minimum value may be identified as “at the time of inspiration”. Further, for example, the average value of each of the feature points P₁ to P₃ is obtained, and a period from the minimum value of the average value to the maximum value may be identified as “the time of expiration”, and the period from the maximum value to the minimum value of the average values may be identified as “the time of inspiration. Further, instead of the total value and the average value, “the time of expiration” and “the time of inspiration” can be determined based on the feature point with the largest variation among the three feature points P₁ to P₃. As described above, by monitoring changes in depth of a plurality of feature points, it is possible to enhance the accuracy of discrimination processing of expiration and inspiration.

However, in the present invention, the change in the depth value of only one feature point may be monitored. In that case, one feature point whose depth can be monitored may be determined from ultrasonic data, and changes in the depth may be monitored. In the case where there is one feature point to be monitored, it is preferable to determine a site having the highest luminance value as a feature point out of M-mode ultrasonic data.

Further, in addition to the identification of “at the time of expiration” and “at the time of inspiration”, the respiration identifying unit 13 can also identify “at the time of apnea” of the subject based on the depth value of each feature point. Specifically, when the depth value of the feature point does not change for a predetermined period (for example, 2 seconds or more) or when the change amount for a predetermined period is a predetermined value or less (for example, 0.5 mm or less), the period without change or with minor change can be identified as apnea. The respiration identifying unit 13 may divide one respiratory cycle into the time of expiration, the time of inspiration, and the time of apnea. Further, the respiration identifying unit 13 divides one respiratory cycle into the time of expiration and the time of inspiration, and when an apnea occurs during the expiration and inspiration, the respiration identifying unit 13 may discriminate whether the apnea occurs during expiration or during inspiration.

The counting unit 14 counts the number of respirations of the subject based on the discrimination of the expiration and the inspiration identified by the respiration identifying unit 13. Specifically, the counting unit 14 counts one repetition of expiration and inspiration as one respiration and counts how many times the respiration has been performed per unit time (for example, per minute). Further, the counting unit 14 can also count the cumulative number of respirations from a certain point in time. Information on the number of respirations per unit time counted by the counting unit 14 or the cumulative number of respirations is accumulated in the storage unit 20. Further, these pieces of information can be displayed on the display.

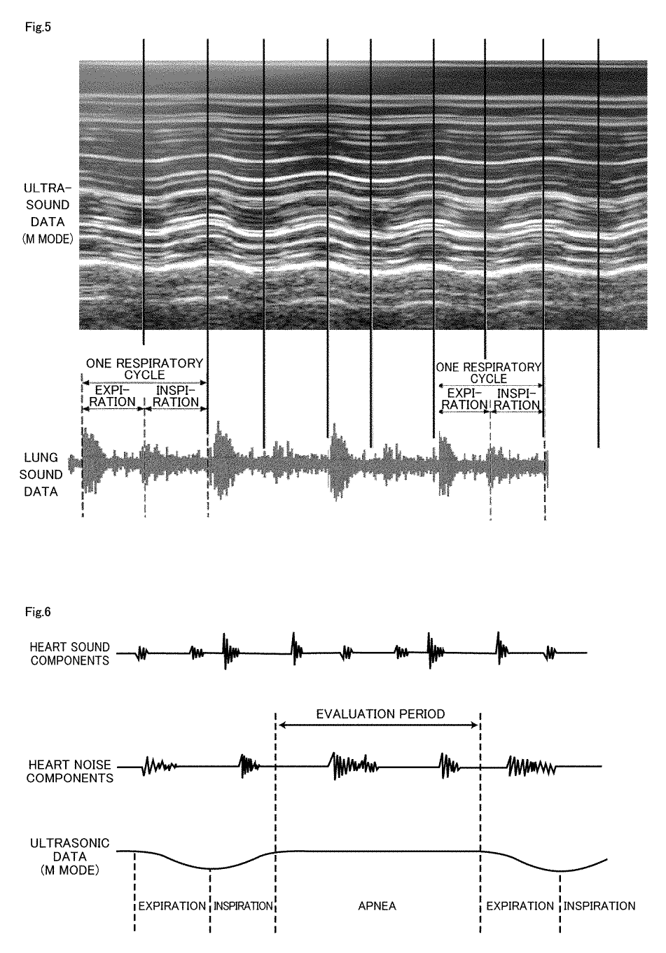

The lung sound analysis unit 15 analyzes lung sound data obtained from the lung sound sensor 3 on the basis of the discrimination information between expiration and inspiration identified by the respiration identifying unit 13. FIG. 5 schematically indicates the process performed by the lung sound analysis unit 15. As illustrated in FIG. 5, the lung sound data obtained from the lung sound sensor 3 is data indicating the amplitude (volume represented by sone or dB) of a sound, and the frequency (Hz) of a sound. However, the lung sound data alone cannot be distinguished between the sound component at the time of expiration and the sound component at the time of inspiration. Therefore, by temporally linking the discrimination information of expiration and inspiration obtained by the respiration identifying unit 13 and the lung sound data obtained from the lung sound sensor 3, sound components at the time of expiration and sound components at the time of inspiration can be extracted from the lung sound data. Thus, the lung sound analysis unit 15 identifies the lung sound at the time of expiration and the lung sound at the time of inspiration based on the lung sound data received from the lung sound sensor 3 and the information on expiration and inspiration identified by the respiration identifying unit 13. The lung sound analysis unit 15 distinguishes information (volume and frequency) on lung sound at the time of expiration and information (volume and frequency) on lung sound at the time of inspiration and stores the information in the storage unit 20. Now that the analysis of the lung sounds by the lung sound analysis unit 15 may be performed each time for each respiratory cycle, or may be performed periodically for each predetermined cycle.

The lung sound determination unit 16 classifies sound components included in lung sound data based on the lung sound at the time of expiration and/or the lung sound at the time of inspiration distinguished by the lung sound analysis unit 15. Classification of lung sounds can be arbitrarily designed, but may be performed according to, for example, a known lung sound classification table (refer to FIG. 7). For example, the table data regarding the classification of lung sounds as indicated in FIG. 7 is stored in the storage unit 20, and the lung sound determination unit 16 refers to the table data of the storage unit 20 and classify automatically the sound components included in the lung sound of the subject. For example, when the lung sound at the time of expiration contains an intermittent sound component of 250 to 500 Hz, the lung sound determination unit 16 includes “discontinuous rale: coarse crackles” in the lung sound of the subject. In addition, for example, when the lung sound at the time of expiration contains a continuous sound component of 400 Hz, the lung sound determination unit 16 can determine that “continuous crackles: wheezes” is included in the lung sound of the subject. Such a determination result by the lung sound determination unit 16 is stored in the storage unit 20 and displayed by a display. As a result, even when performing an automatic diagnosis or when performing a remote diagnosis in which an medical worker does not face a subject, the physician can discriminate the subject's expiration and inspiration, and the abnormality included in the lung sound can be accurately diagnosed.

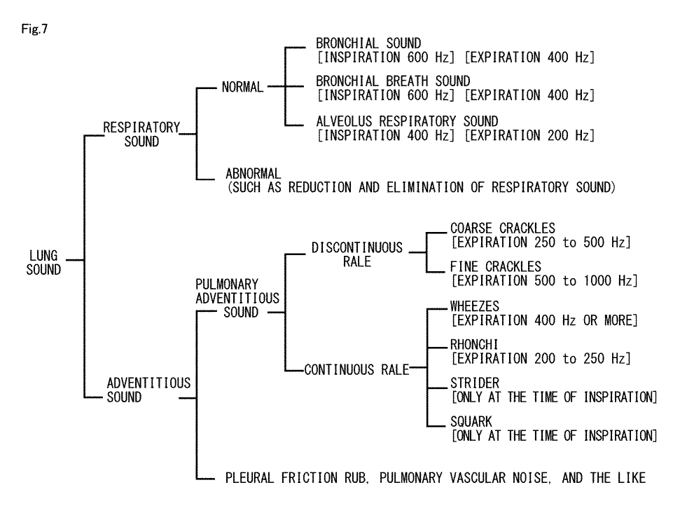

The heart sound analysis unit 17 analyzes heart sound data obtained from the heart sound sensor 4 based on the discrimination information at the time of apnea identified by the respiration identifying unit 13. FIG. 6 schematically indicates the process performed by the heart sound analysis unit 17. As illustrated in FIG. 6, the heart sound analysis unit 17 can classify heart sound data obtained from the heart sound sensor 4 into heart sound components (I sound, II sound, III sound, IV sound) and the other heart murmur components. A known method may be used for the separation processing of the heart sound component and the heart murmur component. Here, when the subject is breathing at the time of acquisition of heart sound data, in addition to actual heart murmur, noise generated by respiration may occur in the heart murmur component separated from the heart sound data. For this reason, when heart murmur components during respiration are evaluated, it may not be possible to properly evaluate only actual heart murmur. Therefore, by evaluating the heart murmur components only at the timing when it is determined that the respiration identifying unit 13 determines that an apnea occurs, it is possible to properly evaluate a heart murmur excluding a respiratory noise. The heart sound analysis unit 17 extracts, from the heart sound data received from the heart sound sensor 4, information (volume and frequency) related to the heart murmur component during apnea identified by the respiration identifying unit 13 and stores the information in the storage unit 20.

The heart sound determination unit 18 receives the information on the heart murmur component during apnea, which the heart sound analysis unit 17 has extracted, and evaluates and determines the heart murmur component. For example, information for determining that there is an abnormality in heart murmur is stored in the storage unit 20. Therefore, the heart sound determination unit 18 collates abnormal condition information stored in the storage unit 20 with the information on the heart murmur component received from the heart sound analysis unit 17 and determines whether or not an abnormality is recognized in the heart murmur. Since the heart murmur component extracted by the heart sound analysis unit 17 is only during apnea, the heart sound determination unit 18 can appropriately evaluate and determine the heart murmur.

Hereinabove, in order to represent the content of the present invention, the embodiments of the present invention have been described with reference to the drawings. However, the present invention is not limited to the above embodiments, and includes modifications and improvements apparent to those skilled in the art based on the matters described in the present specification.

### INDUSTRIAL APPLICABILITY

The present invention relates to a living body monitoring device for monitoring living body information such as respiration and pulsation of a subject. Therefore, the present invention can be suitably applied in the field of medical devices.

### REFERENCE SIGNS LIST

1 Living body monitoring device
2 Ultrasonic sensor
3 Lung sound sensor
4 Heart sound sensor
10 Processing unit
11 Synthesis unit
12 Monitoring unit
13 Respiration identifying unit
14 Counting unit
15 Lung sound analysis unit
16 Lung sound determination unit
17 Heart sound analysis unit
18 Heart murmur determination unit
20 Storage unit
30 Input unit
40 Output unit
50 Communication unit

1. A living body monitoring device, comprising:
a monitoring unit configured to monitor a temporal change in depth of one or a plurality of feature points in a living body based on a signal received from an ultrasonic sensor;
a respiration identifying unit for identifying expiration or inspiration based on information regarding the depth of the feature point; and
a lung sound analysis unit configured to identify both or either a lung sound at the time of expiration and a lung sound at the time of inspiration based on lung sound data received from a lung sound sensor and information regarding expiration or inspiration identified by the respiration identifying unit.

2. (canceled)

3. (canceled)

4. (canceled)

5. (canceled)

6. (canceled)

7. The device according to claim 1, further comprising a lung sound determination unit configured to classify sound components included in the lung sound data based on a lung sound at the time of expiration and/or a lung sound at the time of inspiration identified by the lung sound analysis unit.

8. A living body monitoring device, comprising:
a monitoring unit configured to monitor a temporal change in depth of one or a plurality of feature points in a living body based on a signal received from an ultrasonic sensor;
a respiration identifying unit for identifying the timing of apnea based on information regarding the depth of the feature point; and
a heart sound analysis unit configured to extract heart sounds during an apnea identified by the respiration identifying unit from heart sound data received from a heart sound sensor.
